(19) United States
(12) Patent Application Publication
(10) Pub. No.: US 2014/0363845 A1
(43) Pub. Date: Dec. 11, 2014

(54) CHOLESTEROL-BASED MEDIA SUPPLEMENTS FOR CELL CULTURE

(71) Applicant: Biogen Idec MA Inc., Cambridge, MA (US)

(72) Inventors: Marty Sinacore, Andover, MA (US); Yiwen Tao, San Diego, CA (US); Thomas Ryll, Weston, MA (US); Helena Yusuf-Makagiansar, San Diego, CA (US)

(73) Assignee: Biogen Idec MA Inc., Cambridge, MA (US)

(21) Appl. No.: 14/278,046

(22) Filed: May 15, 2014

Related U.S. Application Data
(63) Continuation of application No. 14/128,900.
(60) Provisional application No. 61/504,096, filed on Jul. 1, 2011.

(51) Int. Cl. C07K 16/28; C12N 5/078

(52) U.S. Cl. CPC C07K 16/2839; C12N 5/0634; C07K 2317/24
USPC 435/69.6; 435/325; 435/355

(57) ABSTRACT

The disclosure provides compositions and methods for high density cell culture and banking of cholesterol auxotrophic cells.

### RELATED APPLICATIONS

This application claims the benefit under 35 U.S.C. §119(e) of U.S. provisional application No. 61/504,096, filed Jul. 1, 2011, the content of which is hereby incorporated by reference in its entirety.

### FIELD OF THE INVENTION

Aspects of the invention are in the field of mammalian cell culture and particularly relate to compositions and methods for culturing cells that are cholesterol auxotrophic in nature.

### BACKGROUND

Cholesterol auxotrophic (cholesterol dependent) cells, such as the myeloma cell line, NS0, are unable to grow in the absence of medium supplemented with cholesterol. For large-scale bioprocesses requiring high cell density growth of cholesterol-dependent cells, polymer-based bioreactors are often used. An irreversible reaction between cholesterol and the polymer, however, often depletes cholesterol from both the supplemented culture medium and the membrane of the cells (see, Kadarusman et al. Biotechnol Prog. 2005; 21:1341-1346; Okonkowski et al. J Biosci Bioeng. 2007; 103:50-59).

### SUMMARY OF THE INVENTION

Provided herein are compositions and methods for high cell density growth and banking of cholesterol-dependent cells.

In some embodiments, aspects of the invention relate to a method of culturing a population of cholesterol auxotrophic cells in a bioreactor using a culture medium that is supplemented with a composition comprising cholesterol associated with a carrier, and free cholesterol, wherein the ratio of free cholesterol to carrier-associated cholesterol is at least 1:5. In some embodiments, the ratio is at least 2:5, at least 3:5, at least 4:5, at least 1:1, at least 2:1, at least 3:1, at least 4:1, at least 5:1, at least 10:1, at least 15:1, or higher.

In some embodiments, aspects of the invention relate to a method that includes providing a population of cholesterol auxotrophic cells; culturing the cells in a bioreactor comprising culture medium supplemented with a composition, wherein the composition includes cholesterol; cyclodextrin; lipids; and ethanol.

In some embodiments, aspects of the invention relate to a method that includes providing a population of cholesterol auxotrophic cells; culturing the cells in a bioreactor comprising culture medium supplemented with a composition that includes cholesterol, cyclodextrin, and ethanol, wherein the ratio of cholesterol:cyclodextrin is about 20:1 to about 1:1.

In some embodiments, aspects of the invention relate to a method that includes providing a population of cholesterol auxotrophic cells; culturing the cells in a bioreactor comprising culture medium supplemented with a composition that includes cholesterol complexed with cyclodextrin, free cholesterol, and ethanol, wherein the ratio of complexed cholesterol:free cholesterol is about 1:12 to about 1:2.

In some embodiments, the ratio of complexed cholesterol:free cholesterol is about 1:8. In some embodiments, the concentration of cholesterol complexed with cyclodextrin is about 2.5 to about 5 mg/L. In some embodiments, the concentration of cholesterol complexed with cyclodextrin is about 2.5 mg/L. In some embodiments, the concentration of free cholesterol is about 10 to about 20 mg/L. In some embodiments, the concentration of free cholesterol (e.g., free synthetic cholesterol) is about 20 mg/L. In some embodiments, the free cholesterol and/or the complexed cholesterol is synthetic cholesterol.

In some embodiments, the complexed cholesterol is complexed with a carrier. In some embodiments, the carrier is a cyclodextrin. In some embodiments, the cyclodextrin is methyl-β-cyclodextrin (mβCD).

In some embodiments, the cholesterol auxotrophic cells are NS0 cells.

In some embodiments, the bioreactor is a disposable bag. In some embodiments, the bioreactor is a polymer-based bioreactor. In some embodiments, the polymer-based bioreactor comprises linear low-density polyethylene (LLDPE). However, it should be appreciated that other polymers may be used.

In some embodiments, one or more lipid(s) also are provided. The lipid(s) can be oleic acid and/or linoleic acid. However, one or more other lipids may be used.

In some embodiments, the cells are cultured to a density of about 2×10⁵ (i.e., about 200,000) viable cells/ml to about 3×10⁷ (i.e., about 30,000,000) viable cells/ml. In some embodiments, the cell viability is about 70% to about 100%. In some embodiments, cell viability of the culture is about 90%.

In some embodiments, the population of cells comprises recombinant cells expressing one or more gene(s) encoding one or more protein(s). In some embodiments, the one or more protein(s) are antibodies (e.g., one or more monoclonal antibodies). In some embodiments, a monoclonal antibody is natalizumab.

In some embodiments, aspects of the invention further comprise collecting the cell culture medium or supernatant (e.g., including cholesterol auxotrophic cells). In some embodiments, cell preparations obtained by the methods described herein are added to one or more storage vials. For example, about 90×10⁶ to about 100×10⁶ viable cells/ml can be added to 5 ml storage vials. However, it should be appreciated that other cell numbers and/or volumes may be used.

In some embodiments, aspects of the invention relate to a composition suitable for growing cholesterol auxotrophic cells. In some embodiments, the composition comprises free cholesterol and carrier-complexed cholesterol in a ratio described herein to be suitable for effective cell growth, for example, in a bioreactor as described herein. In some embodiments, a composition includes cholesterol, cyclodextrin, and/or lipids, and/or an alcohol (e.g., ethanol). In some embodiments, a composition includes cholesterol, cyclodextrin, and an alcohol, (e.g., ethanol), wherein the ratio of cholesterol:cyclodextrin is about 20:1. In some embodiments, a composition includes cholesterol complexed with cyclodextrin, free cholesterol, and an alcohol (e.g., ethanol), wherein the ratio of complexed cholesterol:free cholesterol is about 1:12 to about 1:2. In some embodiments, the ratio of complexed cholesterol:free cholesterol is about 1:8. In some embodiments, the concentration of cholesterol complexed with cyclodextrin is about 2.5 mg/ml to about 5 mg/ml. In some embodiments, the concentration of cholesterol complexed with cyclodextrin is about 2.5 mg/ml. In some embodiments, the concentration of free cholesterol is about 10 mg/ml to about 20 mg/ml. In some embodiments, the concentration of free cholesterol is about 20 mg/ml. It should be appreciated that the free and/or complexed cholesterol can be synthetic cholesterol. In some embodiments, the cyclodextrin is methyl-β-cyclodextrin (mβCD).

In some embodiments, aspects of the invention relate to methods that include dissolving about 10 mg/ml synthetic cholesterol, about 1 mg/ml oleic acid, and about 1 mg/ml linoleic acid in absolute ethanol to form a solution, and adding this solution to a cholesterol complexed with methyl-β-cyclodextrin (mβCD) at a concentration of about 2.2 mg/ml to about 2.5 mg/ml.

These and other aspects of the invention are described in more detail herein and illustrated by one or more non-limiting examples.

### BRIEF DESCRIPTION OF THE DRAWINGS

FIG. 1 is a graph of cholesterol adsorption kinetics in a disposable bioreactor bag.

FIG. 2 is a graph of time-dependent cell growth and viability of cells cultured in medium incubated in a disposable WAVE Bioreactor™ bag. Solid line: cell growth; dotted line: viability. Medium was collected from the bag at 15 min (▪), 4 h (▴), 7.5 h (□) and 22 h (∘) incubation times. Fresh medium () as control.

FIG. 3 is a graph of time-dependent cell growth and viability of cells cultured using the cholesterol-containing compositions described herein. Solid line: cell growth; dotted line: viability. Control (∘) 2.5 mg/L Cholesterol Lipid Concentrate (GIBCO™ INVITROGEN™) (GCLC) supplemented shake flask culture); 2.5 mg/L GCLC supplemented WAVE Bioreactor™ bag culture (0); 2.5 mg/L GCLC+20 mg/L free cholesterol lipid concentrate (CLC) supplemented WAVE Bioreactor™ bag culture (▪); 5 mg/L GCLC cholesterol+10 mg/L CLC cholesterol supplemented WAVE Bioreactor™ bag culture (□); 5 mg/L GCLC cholesterol+20 mg/L CLC cholesterol supplemented WAVE Bioreactor™ bag culture (▴).

FIG. 4 is a graph of a cholesterol depletion time course in batch culture of NS0 cells. The experiment was carried out in 1 L disposable polycarbonate shake flasks in duplicate. Experimental conditions were as follows: 2.5 mg/L GCLC at seed density of 4×10⁵/ml (), 5.0 mg/L GCLC at a seed density of 10×10⁵/ml (▪) and 10 mg/L GCLC at a seed density of 100×10⁵/ml (▴). Cholesterol concentration in spent medium was measured at the time points shown (solid line). Daily cell specific growth rates were calculated for each culture (dotted line).

FIG. 5A and FIG. 5B are graphs of a cholesterol dosing study in high cell density cell culture generated using pseudo-perfusion mode. FIG. 5A demonstrates cell growth, and FIG. 5B demonstrates cell viability. Pseudo-perfusion cultures were established in shake flasks using growth medium supplement with GCLC at the following concentrations: 2.5 mg/L (▪), 7.5 mg/L (▴), 10.0 mg/L (0) to 12.5 mg/L (∘). Full volume medium exchanges were carried out on a daily basis.

FIG. 6 is a graph of cell density and viability using the high density NS0 cell perfusion culture in a WAVE Bioreactor™ bag system. Perfusion cultures were inoculated at a starting density of 0.5×10⁶ viable cells/ml using medium supplemented with 2.5 mg/L GCLC and 20 mg/L CLC (day −1). Perfusion was initiated on Day 0 with a bolus 2.5 mg/L GCLC feed. The daily cholesterol bolus feed was subsequently adjusted according to the cell density of the cultures: 2.5 mg/L at cell densities less than 5×10⁶ cells/ml; 5 mg/L at cell densities of 5-10×10⁶ cells/ml, 7.5 mg/L at cell densities of 10-15×10⁶ cells/ml and 10 mg/L at >15×10⁶ cells/ml. Cell growth (solid lines). Viability (dotted lines). Arrows indicate GCLC feed.

### DETAILED DESCRIPTION OF THE INVENTION

In some embodiments, aspects of the invention relate to compositions and methods for high-density cell culture and banking of cholesterol auxotrophic cells. In particular embodiments, compositions described herein contain cholesterol that is complexed with (forms a complex with) a carrier in an amount sufficient to gain entry into cells and sustain cell viability and growth, and an excess of free (carrier-free) cholesterol. In some embodiments, the ratio of free cholesterol to cholesterol in the form of a cholesterol-carrier complex is above a minimal threshold effective to support the growth of a cholesterol-auxotrophic cell line. In some embodiments, additional agents are provided, for example, to stabilize the free cholesterol. The additional agents can include lipids and/or alcohol (e.g., ethanol). In related embodiment, composition provided herein permit cell cultivation in bioreactors made of polymers that bind cholesterol (e.g., linear low-density polyethylene (LLDPE)). In some instances, a carrier (of the cholesterol complex) is necessary for cholesterol to gain entry into a cell, while excess free cholesterol adsorbs to the polymer, thereby preventing the polymer from sequestering cholesterol/carrier complexes

As used herein, “cholesterol complexed with a carrier” or “carrier-associated cholesterol” may refer to a reversible (e.g., non-covalent) interaction between a carrier and cholesterol. The carrier (e.g., a cyclodextrin) forms a reversible complex with cholesterol and maintains the lipid in a soluble state in aqueous culture media/solution. In this way, the carrier can deliver the otherwise insoluble cholesterol to cells in culture. Once in the cell, the carrier and cholesterol can dissociate. The mechanism is similar to that of serum proteins (such as serum albumin) in blood, which serve as carriers to deliver otherwise insoluble cholesterol to tissues in the body.

Certain embodiments described herein are directed to cholesterol auxotrophic cells. Cholesterol auxotrophs (or cells auxotrophic for cholesterol) require cholesterol for growth, but are unable to synthesize it. Examples of cholesterol auxotrophic cell lines used herein include the murine NS0 myeloma cell line and its derivative cell lines. The NS0 cell line obtained from the European Collection of Cell Cultures (ECACC culture number 85110503) is a mouse myeloma cell line with lymphoblastic morphology. It is a subclone of the NS-1 cell line that is also known to be cholesterol dependent. Other cholesterol auxotrophic cells that may be used herein include Chinese hamster ovary (CHO) cell lines SRD-6, SRD-13A cells, SRD-128 cells, and M19 cells (see, Storey et al. J of Lipid Res. 1997. 38: 711-722; Rawson et al. Mol Cell. 1997. 1:47-57; Sakai et al. 1998 Mol Cell. 2: 505-514; Rawson et al. 1999. J Biol Chem. 274:28549-25556, the disclosures of which are herein incorporated by reference). It should be understood that the compositions and methods described herein can be used to culture any cholesterol auxotrophic cells.

Cholesterol used in the methods and composition described herein may be animal-derived or synthetic. In certain embodiments, cholesterol is complexed with a carrier to gain entry into a cell. Cholesterol that forms a complex with a carrier is referred to herein as “bound cholesterol” or “complexed cholesterol.” Cholesterol carriers include without limitation cyclodextrins, for example, 2-hydroxypropyl-β-cyclodextrin, and methyl-β-cyclodextrin (MβCD). The water-soluble MβCD forms soluble inclusion complexes with cholesterol, thereby enhancing cholesterol solubility in aqueous solution. In particular embodiments described herein, cholesterol forms a complex with (or forms a soluble inclusion complex with) MβCD, while in other embodiments, cholesterol is in its free form. However, it should be appreciated that one or more other cholesterol carriers may be used (e.g., one or more other carriers that form a complex, for example a soluble inclusion complex, with cholesterol).

In particular embodiments, compositions described herein comprise lipids. Examples of lipids include without limitation fatty acyls (e.g., eicosanoids), glycerolipids, glycerophospholipds, sphingolipids, sterol lipids, prenol lipids, saccarolipids, and polyketides. The lipids may be saturated or unsaturated. In particular embodiments, unsaturated fatty acids, oleic acid, linoleic acid, and/or α-linolenic acid are used. One or more other lipids may be used.

In certain embodiments, provided herein are compositions comprising cholesterol complexed to a carrier, such as cyclodextrin, wherein the mass ratio of cholesterol:carrier in the complex is approximately (about) 20:1 to 1:1, or any ratio in between. For example, the ratio of cholesterol:carrier may be approximately 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, or 1:1. In some embodiments, the molar ratio of cholesterol:carrier in the complex is approximately 20:1 to 1:1, or any ratio in between.

Compositions described herein may be stock compositions (e.g., 1000×concentration), and in some embodiments, comprise mixture of water and ethanol as a solvent (or other alcohol or other suitable solvent), or they may be working compositions comprising cell culture medium. For example, a stock composition may comprise approximately 2.5 to 5.0 mg/ml bound cholesterol and approximately 10.0 to 20.0 mg/ml free cholesterol, while a working composition may comprise approximately 2.5 to 5.0 mg/L bound cholesterol and approximately 10.0 to 20.0 mg/L free cholesterol. In certain other embodiments, a stock composition comprises approximately 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/ml bound cholesterol and approximately 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 mg/ml free cholesterol, while a working composition comprises 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 mg/ml bound cholesterol and approximately 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5, 22.0, 22.5, 23.0, 23.5, 24.0, 24.5, 25.0, 25.5, 26.0, 26.5, 27.0, 27.5, 28.0, 28.5, 29.0, 29.5, or 30.0 mg/ml free cholesterol. In particular embodiments, a stock composition comprises approximately 2.5 mg/ml bound cholesterol and approximately 20.0 mg/ml free cholesterol, while a working composition comprises approximately 2.5 mg/L bound cholesterol and approximately 20.0 mg/L free cholesterol.

Also provided herein are ratios of bound cholesterol to free cholesterol, independent of cholesterol to carrier ratio. In certain embodiments, the ratio of bound cholesterol:free (unbound) cholesterol is approximately 1:20 to 1:1, or any ratio in between. For example, the ratio of bound cholesterol:free cholesterol may be approximately 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, or 1:1. In certain embodiments, the ratio of bound cholesterol:free cholesterol is approximately 1:12 to 1:2. In particular embodiments, the ratio is approximately 1:8.

In some embodiments, culture medium used herein may be commercially available and/or well-described (see, Birch J. R. 2000. R. G. Spier (Ed.) Encyclopedia of Cell Technology, Wiley. 2000. pp. 411-424; Keen, M. J. 1995. Cytotechnology. 17: 125-132; Zang, et al. 1995. Bio/Technology. 13: 389-392, the disclosures of which are herein incorporated by reference). In particular embodiments, the culture medium may be protein-free.

Certain embodiments described herein are directed to efficient, high-density cell growth. In any one of the embodiments described herein, the cholesterol auxotrophic cells may be cultured to a density of approximately 1×10⁴ to 1×10⁸ viable cells/ml cell culture medium. In some embodiments, the cells are cultured to a density of approximately, 1×10⁴, 2×10⁴, 3×10⁴. 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶. 1×10⁷, 2×10⁷, 3×10⁷, 4×10⁷, 5×10⁷, 6×10⁷, 7×10⁷, 8×10⁷, 9×10⁷, or 1×10⁸ viable cells/ml. In particular embodiments, the cells are cultured to a density of approximately 2×10⁵ to 3×10⁷ viable cells/ml.

In certain embodiments, cells are cultured in a bioreactor. A bioreactor refers to a container in which cells are cultured, for example, a culture flask, dish, or bag that may be single-use (disposable), autoclavable, or sterilizable. The bioreactor may be made of glass, or it may be polymer-based, or it may be make of other materials. In particular embodiments, the bioreactor is made of linear low-density polyethylene (LLDPE), for example, a LLDPE WAVE Bioreactor™ (GE Healthcare™). One or more compositions as described herein may be used with other polymers or surfaces that are known or determined to be cholesterol-binding.

In some embodiments, a bioreactor refers to a cell culture bioreactor, including a stirred tank (e.g., well mixed) bioreactor or tubular reactor (e.g., plug flow), airlift bioreactor, membrane stirred tank, spin filter stirred tank, vibromixer, fluidized bed reactor, or a membrane bioreactor. The mode of operating the bioreactor may be a batch or continuous processes and will depend on the cell strain being cultured. A bioreactor is continuous when the feed and product streams are continuously being fed and withdrawn from the system. A batch bioreactor may have a continuous recirculating flow, but no continuous feeding of nutrient or product harvest. For intermittent-harvest and fedbatch (or batch fed) cultures, cells are inoculated at a lower viable cell density in a medium that is similar in composition to a batch medium. Cells are allowed to grow exponentially with essentially no external manipulation until nutrients are somewhat depleted and cells are approaching stationary growth phase. At this point, for an intermittent harvest batch-fed process, a portion of the cells and product may be harvested, and the removed culture medium is replenished with fresh medium. This process may be repeated several times. For production of recombinant proteins and antibodies, a fedbatch process may be used. While cells are growing exponentially, but nutrients are becoming depleted, concentrated feed medium (e.g., 10-15 times concentrated basal medium) is added either continuously or intermittently to supply additional nutrients, allowing for further increase in cell concentration and the length of the production phase. Fresh medium may be added proportionally to cell concentration without removal of culture medium (broth). To accommodate the addition of medium, a fedbatch culture is started in a volume much lower that the full capacity of the bioreactor (e.g., approximately 40% to 50% of the maximum volume) (website:hugroup.cems.umn.edu/Cell_Technology/Notes/Cell % 20Culture %20Bioreactors.pdf (accessed on Jun. 1, 2011), the entire content of which is herein incorporated by reference).

In other embodiments described herein, cells are cultured using a perfusion-based high cell density seed train expansion procedure, involving the creation of a high cell density cell bank. The high density cell bank vials are used to directly inoculate a seed train bioreactor, for example, a perfusion WAVE Bioreactor™ (GE Healthcare™) (see, Tao et al. 2011. Biolechnol Frog. 2011. 00(00): 1-6 (published online), the disclosure of which is herein incorporated by reference in its entirety).

Cells of any one of the embodiments described herein may produce antibodies, or antigen-binding fragments, thereof. Some embodiments described herein relate to methods for producing and/or isolating antibodies, or antigen-binding fragments, thereof. As used herein, the term “antibody” refers to a Y-shaped protein used by the immune system to identify and neutralize foreign objects (e.g., bacteria and viruses). In some embodiments, an antibody may be a glycoprotein comprising at least two heavy (H) chains and two light (L) chains inter-connected by disulfide bonds. The term “antigen-binding fragment” of an antibody as used herein, refers to one or more portions of an antibody that retain the ability to specifically bind to an antigen. It has been shown that the antigen-binding function of an antibody can be performed by fragments of a full-length antibody.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known. Monoclonal antibody production may be effected by techniques, which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line. In yet other embodiments, the antibodies can be chimeric or humanized antibodies. As used herein, the term “chimeric antibody” refers to an antibody that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). In certain embodiments, the antibodies are human antibodies. The term “human antibody”, as used herein, is intended to include antibodies having variable and constant regions derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse have been grafted onto human framework sequences (referred to herein as “humanized antibodies”).

Examples of monoclonal antibodies that may be produced by the methods described herein include Abciximab (REOPRO®), Adalimumab (HUMIRA®), Alemtuzumab (CAMPATH®), Basiliximab (SIMULECT®), Bevacizumab (AVASTIN®), Cetuximab (ERBITUX®), Certolizumab pegol (CIMZIA®), Daclizumab (ZENAPAX®), Eculizumab (SOLIRIS®), Efalizumab (RAPTIVA®), Gemtuzumab (MYLOTARG®), Ibritumomab tiuxetan (ZEVALIN®), Infliximab (REMICADE®), Muromonab-CD3 (ORTHOCLONE OKT3®), Natalizumab (TYSABRI®), Omalizumab (XOLAIR®), Palivizumab (SYNAGIS®), Panitumumab (VECTIBIX®), Ranibizumab (LUCENTIS®), Rituximab (RITUXAN®), Tositumomab (BEXXAR®), and Trastuzumab (HERCEPTIN®). In particular embodiments, the cholesterol auxotrophic cells produce humanized monoclonal antibodies, for example, Natalizumab (TYSABRI®). Natalizumab can be used to treat multiple sclerosis and Crohn's disease.

In certain embodiments, therapeutic monoclonal antibodies are produced using the Glutamine Synthetase (GS) Gene Expression System (Lonza Biologics). GS synthesizes glutamine from glutamate and ammonium. Because glutamate is an essential amino acid, transfection of cells that lack endogenous GS (e.g., NS0 cells), with the GS vector confers the ability to grow in glutamine-free medium.

In certain embodiments, provided herein are methods comprising providing a population of cholesterol auxotrophic cells, and culturing the cells in a bioreactor comprising culture medium supplemented with a composition comprising: cholesterol, a carrier, lipids, and ethanol. In some embodiments, provided herein are methods comprising providing a population of NS0 cells, and culturing the cells in a bioreactor comprising culture medium supplemented with a composition comprising: cholesterol, a carrier, lipids, and ethanol. In other embodiments, provided herein are methods comprising providing a population of cholesterol auxotrophic cells, and culturing the cells in a bioreactor comprising culture medium supplemented with a composition comprising: synthetic cholesterol, a carrier, lipids, and ethanol. In some embodiments, provided herein are methods comprising providing a population of cholesterol auxotrophic cells, and culturing the cells in a bioreactor comprising culture medium supplemented with a composition comprising: cholesterol, cyclodextrin, lipids, and ethanol. In certain embodiments, provided herein are methods comprising providing a population of NS0 cells, and culturing the cells in a bioreactor comprising culture medium supplemented with a composition comprising: synthetic cholesterol, mβCD, lipids, and ethanol. In yet other embodiments, provided herein are methods comprising providing a population of cholesterol auxotrophic cells, and culturing the cells in a bioreactor comprising culture medium supplemented with a composition comprising: synthetic cholesterol complexed with cyclodextrin, free cholesterol, lipids, and ethanol. In still other embodiments, provided herein are methods comprising providing a population of NS0 cells, and culturing the cells in a bioreactor comprising culture medium supplemented with a composition comprising: synthetic cholesterol complexed with cyclodextrin, free cholesterol, lipids, and ethanol.

Also provided herein are methods of formulating a composition, comprising combining an aqueous concentrate of cholesterol complexed with a carrier with free cholesterol and lipids that have been dissolved in ethanol (e.g., absolute ethanol).

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove.

### EXAMPLES

### Example 1

### Kinetics of Cholesterol Adsorption in Disposable Bioreactor Bags

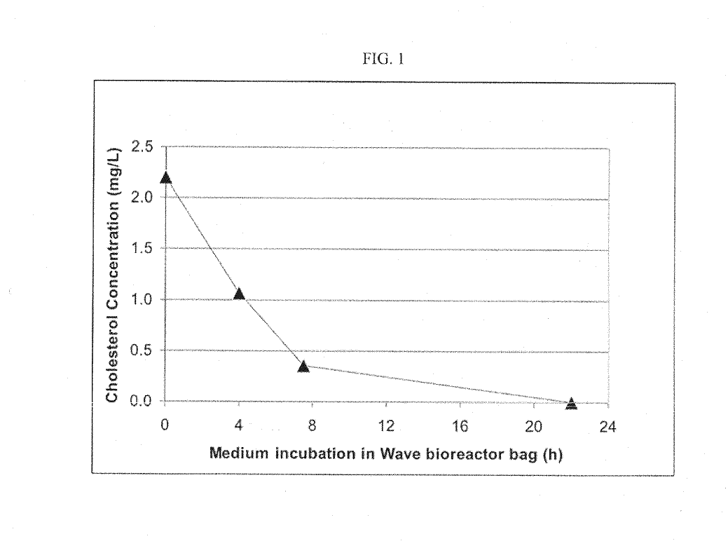

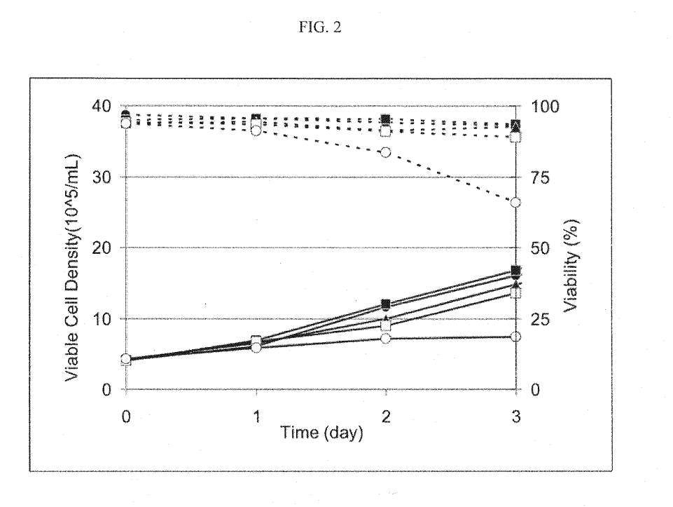

The rate of cholesterol depletion from the culture medium in single-use disposable bioreactor bags was determined. A medium hold study was performed in which 1.4 L of fresh pre-warmed culture medium containing 2.5 mg/L cholesterol (1 ml GCLC/liter medium) was added into a 2 L WAVE BIOREACTOR™ bag. The bag was then incubated under standard culture conditions, and 200 ml medium aliquots were aseptically removed from the bag at 15 min, 4 h, 7.5 h and 22 h incubation times. The medium aliquots at each time point were analyzed for cholesterol concentration (FIG. 1) and used to inoculate shake flask cultures seeded at 4×10⁵ cells/ml. Shake-flask cultures were passaged for three days under standard conditions. A shake-flask control culture (no prior exposure to WAVE Bioreactor™ bag) containing 2.5 mg/L cholesterol (1 ml GCLC per liter medium) was seeded in parallel. Viable cell density and viability were measured every 24 hours (FIG. 2).

The medium hold study indicated that the cholesterol concentration in the medium declined to below detectable limits after 22 hours of incubation in the WAVE Bioreactor™ bag. The growth and viability data from shake flask cultures (FIG. 2) showed that there was a corresponding decrease in the ability of the medium to support cell growth. Based on these data we decided that the cholesterol concentration in the medium should be maintained above 1.0 mg/L to support cell growth.

### Example 2

### Cholesterol Supplement Optimization for Cultivation of NS0 Cells

Cells were suspended in fresh medium containing increased amount of GCLC at concentrations of 5 mg/L and 7.5 mg/L and then incubated for 3 days in 2 L bags under standard cell culture conditions. Under these conditions cell growth was below that observed in control shake flask cultures. The effect of the ethanol-based cholesterol-lipid concentrate stock solution (CLC) at a dose range of 10 mg, 20 mg, 30 mg, and 40 mg cholesterol per liter medium was assessed. None of the conditions yielded cell growth comparable to shake flask control cultures. An ethanol dose study indicated that cell growth was not inhibited at ethanol concentrations below 2%, indicating that ethanol toxicity was not a significant factor.

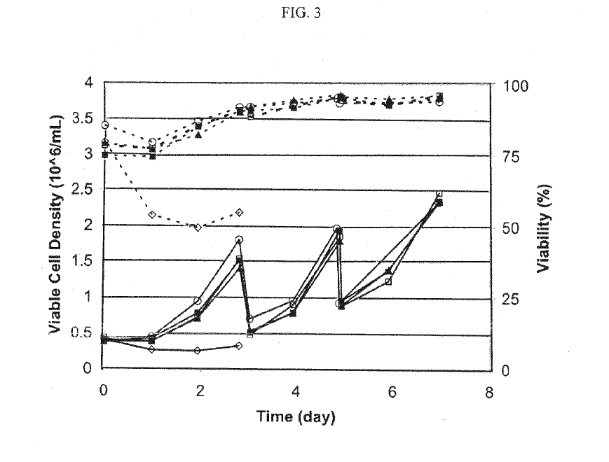

NS0 cultures were inoculated in 2 L WAVE BIOREACTOR™ bioreactor bags using medium supplemented with various combinations of GCLC and CLC. The cell cultures were scaled up in the WAVE Bioreactor™ bags on day 3 and day 5 post inoculations by adding additional medium supplemented with the same concentration of CLC. The viable cell density was measured daily. The data in FIG. 3 indicated that each tested combination of cholesterol concentrations, ranging 2.5-5.0 mg/L GCLC cholesterol and 10-20 mg/L CLC cholesterol, supported cell growth comparable to the control shake flask. GCLC supplement alone did not support cell growth in bag cultures.

### Example 3

### Cholesterol Bolus Feed Optimization Under High Cell Density Perfusion Culture

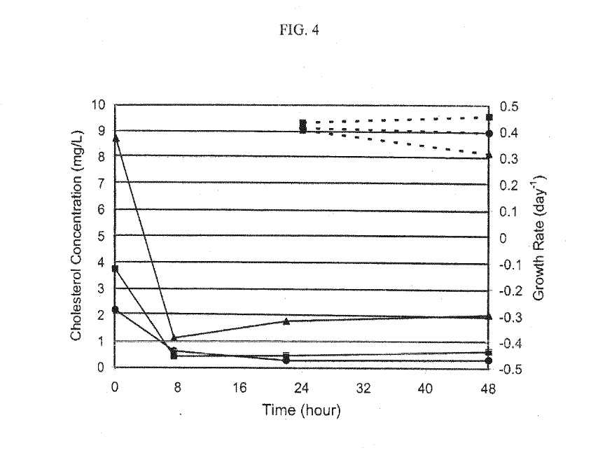

High cell density perfusion culture presented a different challenge with respect to cholesterol delivery. A bolus-feed strategy was tested and used to provide the required levels of cholesterol to high cell density NS0 cultures. To better understand cholesterol requirements under high cell density conditions, we examined cholesterol depletion kinetics. Accordingly, experiments were performed in 1 L polycarbonate shake flasks in which both the seed density was varied from 4×10⁵/ml to 100×10⁵/ml and the GCLC cholesterol supplement was varied from 2.5-10.0 mg/L. Cell free medium samples were collected at 15 min. 7.5 h, 22 h, and 48 h post inoculation and analyzed for cholesterol concentration. Culture duration was restricted to 2 days to prevent depletion of other key nutrients that may have limited the cultures at high cell densities. Cell growth was monitored every 24 hours. The data shown in FIG. 4 indicated that cholesterol depletion was related to the initial seed density of the cultures. Furthermore, in all cultures, the cholesterol concentrations bottomed out at the 7.5 h time point.

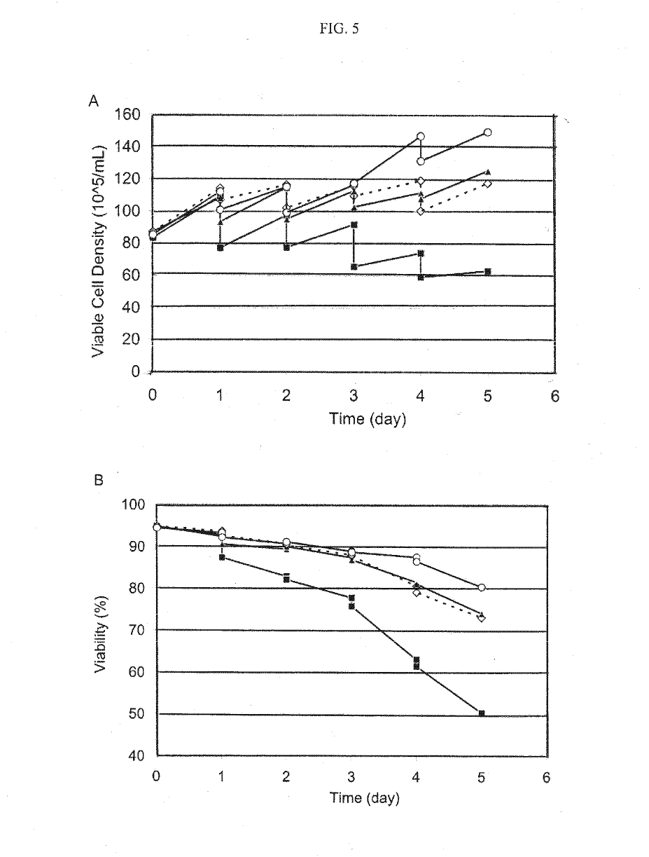

Cholesterol delivery using a high cell density pseudo-perfusion culture mode in shake flasks cultures was optimized. There was full volume exchange of medium in the shake flasks on a daily basis. The initial seed density was 8×10⁶ cells/ml, and one volume of fresh growth medium was exchanged daily starting on day 1 post inoculation. The cholesterol concentration in the cultures ranged from 2.5 mg/L to 12.5 mg/L. The data shown in FIG. 5 indicated that 2.5 mg total cholesterol/L was insufficient to support growth of cells in high cell density pseudo-perfusion culture and that >7.5 mg/L was required.

Results indicated that a daily bolus feed strategy was feasible to support cell growth under high cell density perfusion culture conditions. Cholesterol feeds should be increased as cell density increases. Accordingly, the NS0 cells were inoculated in perfusion WAVE Bioreactor™ using medium supplemented with 2.5 mg/L GCLC and 20 mg/L CLC. Medium perfusion was started the day after inoculation at a cell specific perfusion rate (CSPR) of 0.5 nl/cell/day. Daily cholesterol bolus feeds were initiated at the start of perfusion using an initial GCLC feed of 2.5 mg/L and increased daily according to cell growth.

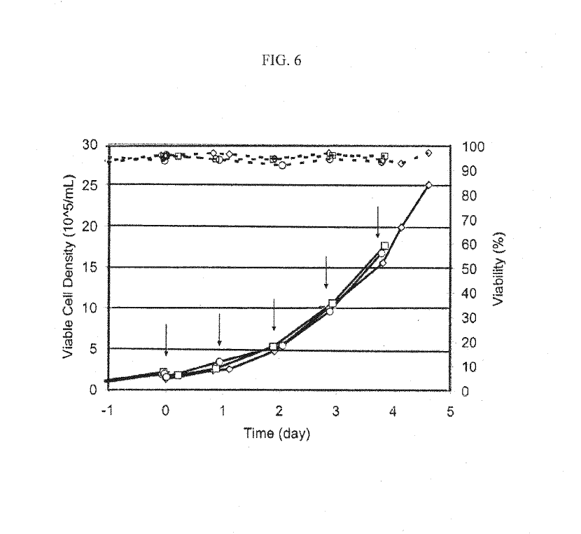

FIG. 6 shows the result of three independent perfusion cultures. Peak cell densities of 16-25×10⁶ viable cells/ml were achieved while maintaining cell viability at approximately 90%. During perfusion culture, nutrient levels were also monitored, and there was no indication of glucose or glutamate depletion.

### Example 4

### Evaluation of High Density Cell Banks

Cells were harvested from high cell density perfusion cultures and staged for cell banking. Cells were pelleted by centrifugation and resuspended in cryopreservative medium (growth medium supplemented with 10% dimethyl sulfoxide) at a target cell density of 100×10⁶ viable cells/ml in 4.5 ml aliquots. Upon completion of vialing, the cell banks were frozen in a controlled rate freezer before transfer to a vapor phase liquid nitrogen freezer for long term storage.

HD-cell banks were evaluated by thawing in a 37° C. water bath and directly inoculated into a 20 L WAVE BIOREACTOR™ using pre-warmed medium supplemented with 2.5 mg/L GCLC and 10 mg/L CLC at an initial working volume of 1 L. The working volume was expanded to 10 L in two subsequent steps. 2.5 mg/L cholesterol from GCLC and 10 mg/L from CLC were supplemented in pre-warmed medium before addition to the WAVE BIOREACTOR™. FIG. 7 shows the thaw and recovery data using vials from six HD cell banks. Desired thaw and recovery of HD cell bank was achieved in LLDPE WAVE BIOREACTORS™. Directly thawing HD WCB vial in disposable bioreactor shortened 14 days during seed train expansion.

### Materials and Methods

### Cell Line and Culture Condition

A cholesterol-dependent NS0 cell line expressing recombinant antibody was used in the study. The culture medium used was a chemically defined medium formulation. NS0 cell cultures were maintained in low cell density shake flask cultures using growth medium supplemented with Cholesterol Lipid Concentrate (GCLC, 1.0 ml/L) (GIBCO™ INVITROGEN™). Cultures were seeded at 3-4×10⁶/ml in Corning 500 ml or 1 L polycarbonate shake flasks fitted with vented caps and agitated at 125 rpm in a non-humidified incubator at 36.5° C. in a 5% CO₂:95% air atmosphere.

While using disposable bioreactors, the bags were rocking at 15 rpm (1-L working volume) increased to 18 rpm (2-5 L working volume) and 25 rpm when 10 L full working volume reached. The rocking angle was fixed at 7.5°. Cells were cultivated at 36.5° C. with continuous overlay gas flow rate at 0.1 L/min of 5% CO₂:95% air mixture.

### Cholesterol Supplements

GIBCO™ Cholesterol Lipid Concentrate (GCLC, 1000× Aqueous Liquid) was purchased from INVITROGEN™ (Product Code 0010025DG; Formula No. 04-0042DK). The cholesterol concentration was verified by GC analysis to range from 2.2-2.5 mg/ml. An ethanol based cholesterol-lipid concentrate stock solution (CLC) was prepared by dissolving 10 mg/ml synthetic cholesterol (Invitrogen 01-5089). 1 mg/ml oleic acid (Sigma 01008) and 1 mg/ml linoleic acid (Sigma L1376) in absolute ethanol.

### WAVE Bioreactor™ Bags and System

The disposable bioreactor bags were purchased from GE Healthcare. Three types of bags were used, CB0002L10-01, CB0020L10-01 and CB0020L10-04 (perfusion bag equipped with an interior floating filter as a cell retention device). The bags' contact film was ethylene vinyl acetate/low density polyethylene, a type of copolymer routinely used for blood collection and handling of biological fluids. Outer layers were made of composites that provide strength and low gas permeability.

A WAVE BIOREACTOR™ System (Model 20/50EH) equipped with supervisory control/data acquisition modules (operated by PCDAQ/SCADA software), an on-demand 02MIX20 oxygen/air controller and dissolved oxygen probe (GE HealthCare 28-4116-72) were used. The PC Supervisory control module allowed for dissolved oxygen (DO) control with O₂ gas supplied in the headspace or by varying rocking speed. Medium perfusion was controlled using a weight-based perfusion controller (LOADCELL20 Perfusion Controller) to maintain a constant working volume at a desired perfusion rate. Dissolved oxygen (DO) was controlled at a set point of 30% air saturation. The O₂ concentration in the headspace was automatically regulated by a PC Supervisory control module in a range from 20% to 50%. The culture pH was controlled by manually regulating the CO₂ concentration and the gas flow rate in the headspace. Target pH range was between 6.8 and 7.3.

The culture perfusion rate (volume of fresh medium/working volume of reactor/day, vvd) was increased daily according to the integral cell growth (ICG) of the culture and a cell specific perfusion rate (CSPR) using the following equation.

Perfusion rate(vvd)=CSPR×ICG

Where CSPR (nl medium/cell/day) represents the volume of medium provided to one cell in one day. ICG (10⁶ cells/ml×day) is determined from the area under the viable cell curve of current viable cell density and predicted viable cell density in 24 h interval, which is estimated by the specific cell growth rate of the cell line (see, Tao et al. 2011. Biotechnol Frog. 2011. 00(00): 1-6 (published online)).

### Cell Banking

Cultures were harvested from the perfusion bioreactor at a viable cell density target range of 16-27×10⁶ cells/ml. Cells were pelleted by centrifugation at 700 rpm for 12 minutes at room temperature. Cell pellets were resuspended in cryopreservative medium (growth medium supplemented with 10% dimethyl sulfoxide, Sigma D2650) at a target cell density of 100×10⁶ viable cells/ml. NUNC™ 5 ml CRYOTUBE® vials (NUNC™ 379146) were manually filled with 4.5 ml cell suspensions using a pipeline dispenser (Essen #4355) actuated with a foot switch. Upon completion of vialing, the cell banks were frozen in a controlled rate freezer (Planer Cryo 560-16) before transfer to a vapor phase liquid nitrogen freezer.

Recovery of cryopreserved cell banks was evaluated by thawing individual vials in a 37° C. water bath and inoculating into a 20 L WAVE BIOREACTOR™. The contents of the cell bank vial was resuspended in 1 L of pre-warmed growth medium supplement with GCLC (2.5 mg/L) and CLC (20 mg/L) and then transferred into the WAVE BIOREACTOR™ at an initial working volume of 1.0 L. The working volume was subsequently scaled up to 10 L working volume in the same bioreactor by two separate medium additions on day 3 and day 5 maintaining an initial target seed density of 4.5×10⁵ viable cells/ml after first medium addition.

### Analytical Methods

Viable cell density was determined using a Cedex Automated Cell Culture Analyzer (Roche Innovatis AG). pH and pCO₂ were measured off-line using a BioProfile pHOx analyzer (Nova Biomedical). Cholesterol concentration was measured using GC analysis by Invitrogen Medium Analytical Services.

### EQUIVALENTS AND SCOPE

Those skilled in the art will recognize or be able to ascertain using no more than routine experimentation many equivalents to the specific embodiments described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviations, per the practice in the art. Alternatively, “about” can mean a range of up to 20%, up to 10%, up to 5%, or up to 1% of a given value. Alternatively, the term can mean within an order of magnitude, for example, within 5-fold, or within 2-fold, of a value. Where particular values are described in the application and claims, unless otherwise stated the term “about” meaning within an acceptable error range for the particular value should be assumed.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the methods of the invention can be excluded from any one or more claims, for any reason, whether or not related to the existence of prior art.

What is claimed is:

1. A method, comprising:
culturing a population of cholesterol auxotrophic cells in a bioreactor that contains culture medium supplemented with a composition, the composition comprising
cholesterol associated with a carrier, and
free cholesterol,
wherein the ratio of free cholesterol to carrier-associated cholesterol is at least 1:5.

2-3. (canceled)

4. A method, comprising:
providing a population of cholesterol auxotrophic cells; and
culturing the cells in a bioreactor comprising culture medium supplemented with a composition, the composition comprising (a) cholesterol, cyclodextrin, lipids and ethanol, or (b) cholesterol complexed with cyclodextrin, free cholesterol and ethanol, wherein the ratio of complexed cholesterol:free cholesterol is about 1:12 to about 1:2.

5. The method of claim 4, wherein the ratio of complexed cholesterol:free cholesterol is about 1:8.

6. The method of claim 4, wherein the concentration of cholesterol complexed with cyclodextrin is about 2.5 to about 5 mg/L.

7. (canceled)

8. The method of claim 4, wherein the concentration of free cholesterol is about 10 to about 20 mg/L.

9. (canceled)

10. The method of claim 4, wherein the free cholesterol is synthetic cholesterol.

11. The method of claim 4, wherein the cholesterol in (i) is synthetic cholesterol.

12. The method of claim 4, wherein the cyclodextrin is methyl-β-cyclodextrin (mβCD).

13. The method of claim 4, wherein the cholesterol auxotrophic cells are NS0 cells.

14. The method of claim 4, wherein the bioreactor is a disposable bag.

15. The method of claim 4, wherein the bioreactor is a polymer-based bioreactor.

16. The method of claim 15, wherein the polymer-based bioreactor comprises linear low-density polyethylene (LLDPE).

17. The method of claim 4, further comprising one or more lipid(s).

18. The method of claim 17, wherein the one or more lipid(s) is/are selected from oleic acid and linoleic acid.

19. The method of claim 4, wherein the cells are cultured to a density of about 2×10⁵ viable cells/ml to about 3×10⁷ viable cells/ml.

20. The method of 19, wherein the cell viability is about 70% to about 100%.

21. (canceled)

22. The method of claim 4, wherein the population of cells comprises recombinant cells expressing one or more gene(s) encoding one or more protein(s).

23. The method of claim 22, wherein the one or more protein(s) is a monoclonal antibody.

24. The method of claim 23, wherein the monoclonal antibody is natalizumab.

25-26. (canceled)

27. Cell culture medium or supernatant produced by the method of claim 1.

28-29. (canceled)

30. A composition comprising cholesterol, cyclodextrin, lipids and ethanol.

31-40. (canceled)

41. A method, comprising:
dissolving about 10 mg/ml synthetic cholesterol, about 1 mg/ml oleic acid, and about 1 mg/ml linoleic acid in absolute ethanol to form a solution; and
adding to the solution cholesterol complexed with methyl-β-cyclodextrin (mβCD) at a concentration of about 2.2 to about 2.5 mg/ml.
